(19) United States
(12) Patent Application Publication
(10) Pub. No.: US 2026/0096004 A1
(43) Pub. Date: Apr. 2, 2026

(54) PLASMA TREATMENT ARRANGEMENT

(71) Applicant: CINOGY GMBH, Duderstadt (DE)

(72) Inventors: Dirk WANDKE, Heilbad Heiligenstadt (DE); Ronny LETTKE, Duderstadt (DE); Sarah SCHULZE, Lindau (DE)

(21) Appl. No.: 19/108,966

(22) PCT Filed: Sep. 20, 2023
(86) PCT No.: PCT/EP2023/075971

§ 371 Date: Mar. 5, 2025

(30) Foreign Application Priority Data
Sep. 20, 2022 (DE) DE 102022124101.7

(51) Int. Cl. H05H 1/24

(52) U.S. Cl. CPC H05H 1/2418; H05H 2240/00; H05H 2242/22; H05H 2245/34

(57) ABSTRACT

The invention relates to a plasma treatment arrangement (10) for forming a dielectric barrier plasma discharge having an electrode arrangement (11) and a high-voltage generator, wherein the electrode arrangement (11) has at least one electrode (13) and one dielectric in which the electrode (13) is embedded, said dielectric fully covering the electrode (13) up to a surface (23) which is to be treated, wherein the high-voltage generator (25) uses at least one transformer (17a, 17b) to generate a high AC voltage (U₁₃ₐ, U_{13b}) from an input voltage (U_{E}) which is fed to the high-voltage generator (25), and feeds said high AC voltage to the electrode (13) of the electrode arrangement (11) in order to form a dielectric barrier plasma discharge, wherein there is provision for a safety device (20) which comprises a sensor arrangement (27) having at least one magnetic field sensor (27) for detecting the electromagnetic alternating field generated by the transformer (17a, 17b) and which has an evaluation unit (22), by means of which the electromagnetic alternating field detected by the magnetic field sensor (27) is assigned to one of at least two functional states.

The invention relates to a plasma treatment arrangement for forming a dielectric barrier plasma discharge with an electrode arrangement and a high-voltage generator.

Treating surfaces, such as a human or animal skin surface, with a plasma is a known process. A dielectric barrier plasma discharge is generated under atmospheric pressure by applying an alternating high voltage to an electrode that is embedded in a dielectric and thus shielded from the surface to be treated. Such a treatment device is known, for example, from DE 103 24 926 B3. It can be provided for that the body forms the ground electrode with the surface to be treated. The plasma treatment supports healing of the wound and, in particular, serves to disinfect the skin of human or animal bodies.

A similar treatment device is also known from DE 10 2016 118 569 A1 where the electrode arrangement for forming a dielectric barrier plasma discharge has at least two partial electrodes that are arranged next to each other and insulated from one another by the dielectric. The partial electrodes are fed by means of a control device with partial alternating high voltages that compensate each other with regard to the waveform and the voltage level, whereby the surface to be treated also serves as a ground electrode here.

To be able to adapt to varying skin contours, it is also known to design the dielectric and the electrode embedded by the dielectric of such a plasma treatment device to be flexible. For example, DE 10 2016 108 450 A1 discloses an electrode arrangement in which the dielectric and the electrode are made of a flexible plastic material, the electrode being equipped with additional electrically conductive components.

If the skin surface to be treated is used as a ground electrode for the dielectric barrier plasma discharge, an important aspect is to ensure that there is no unimpeded current in the patient's body when the electrode of the electrode arrangement is supplied with an alternating high voltage to generate the dielectric barrier plasma. Such a current can occur, for example, if the dielectric, which lies on and contacts the skin surface for the purpose of plasma treatment, is so damaged in the area of the electrode that a spark or corona discharge occurs in the direction of the surface of the patient's skin while the alternating high voltage is being supplied. The alternating high voltage required for the dielectric barrier plasma discharge lies within a range of 12 kV to 25 kV, for example, wherein it is important to ensure secure protection against accidental contact.

DE 20 2003 003 133 U1 describes a container that contains two electrodes for generating ozone for sterilizing foodstuffs held in the container. The container has a sensor which can be used to determine whether a lid of the container is securely closed. Only when this is the case can a voltage be applied to the electrodes.

DE 10 2018105 895 A1 describes a plasma generator for generating atmospheric pressure plasma where the generated magnetic field is identified by means of a field probe. Using the measured magnetic field, the piezoelectric transformer can be controlled at its natural frequency by a control unit, which increases the efficiency of plasma generation.

DE 10 2017105 430 A1 discloses a device for generating a non-thermal atmospheric pressure plasma that uses two housings, namely for the transformer and the control circuit. This prevents wear of the control circuit caused by the generated reactive gases.

DE 10 2009 011 960 A1 describes a method for monitoring dielectric barrier plasma discharges during which the electrical energy generated in the medium by the AC voltage applied to the electrodes is measured and the signal components determined which lie above a frequency that can be preset. Said signal components are compared with a reference and the plasma generation process thus regulated.

DE 20 2020 104 271 U1 discloses a device for generating a gas discharge inside a treatment instrument containing a security device that is configured to measure a voltage and/or a frequency of high-frequency electrical pulses at the output side of the transformer. If the measured values are beyond the predetermined limit value, the supply of energy to the transformer is interrupted.

EP 3 796 362 A1 describes a method for generating a plasma in a plasma chamber during which a plasma parameter is measured and used to regulate the excitation voltage.

It is therefore the problem of the present invention to provide an improved plasma treatment arrangement for forming a dielectric barrier plasma discharge with which, in particular, an error or malfunction of the electrode arrangement can be determined.

According to the invention, the problem is solved with the plasma treatment arrangement in accordance with claim 1. Advantageous embodiments of the invention are to be found in the corresponding sub-claims.

According to claim 1, a plasma treatment arrangement for forming a dielectric barrier plasma discharge on a surface to be treated with an electrode arrangement and a high-voltage generator is proposed, the electrode arrangement comprising at least one electrode and a dielectric at least partially embedding the electrode, said dielectric completely covering the electrode towards the surface to be treated. In particular, such a plasma treatment arrangement according to the preamble can be configured in such a way that the surface to be treated acts as a ground electrode.

It is beneficial if the electrode arrangement is designed to be applied to the surface to be treated. The surface to be treated is preferably a human or animal body. Preferably, the electrode arrangement is designed to adapt to a surface contour of the human or animal body. In particular, the electrode arrangement is designed to fit snugly against the human or animal body.

The plasma treatment arrangement preferably includes an attachment device for attaching the electrode arrangement to the human or animal body.

The electrode arrangement is preferably flexible. For example, the dielectric is silicone. The at least one electrode can be designed as a metallization or a dielectric that has been rendered conductive, for example conductive silicone.

The dielectric forms an application side with which the electrode arrangement of the plasma treatment arrangement can be placed on the surface to be treated. The application side of the dielectric can be structured so as to form gas spaces in which the plasma can be generated when the dielectric lies flat with its application side on the surface to be treated. For example, it may be provided that the dielectric has spacers on the contact side in the surface and/or on the edge, between which the gas spaces are formed. However, it is also conceivable to place the electrode arrangement on an (open-pored) textile structure in order to form a distance to the surface to be treated, whereby the plasma can then form in the (open-pored) textile structure.

The electrode arrangement can be or is mechanically connected and electrically contacted to the high-voltage generator so that the electrode arrangement can be separated from the high-voltage generator if necessary (e.g. when replacing the electrode arrangement). To this end, the mechanical connection and electrical contacting are designed to be detachable, e.g. using a known plug connection. Alternatively, it is also conceivable, for example, that the electrode arrangement is firmly connected to the high-voltage generator, meaning that a non-destructive separation of the electrode arrangement from the high-voltage generator is not possible. This is practical, for example, when the high-voltage generator is arranged on or in the dielectric.

An input voltage is supplied to the high-voltage generator, wherein the high-voltage generator generates an alternating high voltage from the input voltage by means of at least one transformer and feeds said alternating high voltage to the electrode of the electrode arrangement for forming a dielectric barrier plasma discharge.

According to the invention, it is now provided for that the plasma treatment arrangement has a safety device that contains a sensor arrangement with at least one magnetic field sensor for detecting the alternating electromagnetic (stray) field generated by the transformer and comprises an evaluation unit, by means of which the alternating electromagnetic (stray) field detected by the magnetic field sensor is assigned to one of at least two functional states. Specifically, the first functional state is a target state and the second functional state is an error state. The evaluation unit is configured to connect the supply of the alternating high voltage to the electrode arrangement when the error state is detected.

Preferably, the sensor arrangement is not used to regulate the alternating high voltage.

The alternating electromagnetic field generated by the transformer is thus detected with the aid of the at least one magnetic field sensor of the safety device. The resulting measured values from the measurement of the alternating electromagnetic field of the transformer are then provided to the evaluation unit e.g. via a measured value interface, the evaluation unit being configured to determine a functional state of the plasma treatment arrangement as a function of the detected measured values.

The strength of the magnetic field can be determined in order to detect the alternating magnetic field generated in that, for example, the magnetic field sensors measure the magnetic flux density. The connection to the magnetic field strength can be established via the permeability u which, among other aspects, takes the material properties of the surrounding medium into account.

With regard to the functional state, the evaluation unit can differentiate between a normal state (operation) and at least one error state and, depending on the measured values of the alternating electromagnetic field detected, determine a normal state or at least one error state as a functional state.

With the present invention, it is possible to detect, in particular, an error state on the basis of the alternating electromagnetic field of the transformer in order to increase operational safety during use of such a plasma treatment arrangement. For example, it is conceivable that, when an error state is detected, the safety device—by way of the evaluation unit—brings the entire plasma treatment arrangement into a secure state.

Based on empirical findings, it can be seen that different functional states (in particular different error states and error-free operation) of such a plasma treatment arrangement in relation to the electrode arrangement can be derived from a detection of the alternating electromagnetic field of the transformer, since different functional states result in a corresponding characteristic of the detected alternating electromagnetic field.

The evaluation of the alternating electromagnetic field detected and detection of parameters and/or a characteristic of the alternating electromagnetic field allows a corresponding functional state to be concluded. The respective predetermined parameters and/or characteristics of the alternating electromagnetic field are allocated to the various functional states of the plasma treatment arrangement, so that the corresponding functional state can be determined as a function of the alternating electromagnetic field detected or its measured values and the predetermined parameters and/or characteristics of the alternating electromagnetic field.

For example, it is conceivable that the alternating electromagnetic field detected is compared with the predetermined parameters and/or characteristics and a corresponding functional state is then determined as a function of the comparison.

According to one preferred embodiment, it is intended that the evaluation unit is configured to determine an error state as a functional state in such a way that a short circuit within the electrode of the electrode arrangement, said electrode comprising at least two partial electrodes, a lack of electrical contacting of the high-voltage generator to the electrode arrangement and/or a defect of the dielectric is determined as a function of the alternating electromagnetic field detected.

In this way, a short circuit between the two partial electrodes, preferably in the area of the connecting part that connects the electrode arrangement to the source of the alternating high voltage, can be determined on the basis of the alternating electromagnetic field detected or the measured values of the at least one magnetic field sensor in an electrode arrangement with at least two partial electrodes.

Furthermore, on the basis of the alternating electromagnetic field detected in such an electrode arrangement with at least two partial electrodes, it is possible to determine a two-sided defect of the dielectric when a short circuit occurs between the two partial electrodes due to a defect of the dielectric. However, it is also conceivable that only a one-sided defect of the dielectric is detected, i.e. only in the area of one partial electrode or in the area of the electrode without partial electrodes, by way of the alternating electromagnetic field detected. For example, it can be detected from the parameters and/or the characteristic when a short circuit with the ground electrode occurs due to the defect in the dielectric.

Furthermore, on the basis of the alternating electromagnetic field detected it is possible to determine the lack of electrical contacting of the high-voltage generator to the electrode arrangement, for example when the connection arrangement does not electrically contact the electrode embedded in the dielectric properly in the area of the connecting piece. Such an idle state can effect a subsequent corona discharge, for example, wherein the accompanying characteristic of the alternating electromagnetic field detected enables this error state to be recognized.

Accordingly, one embodiment provides that the evaluation unit is configured to determine a functional state of the plasma treatment arrangement from a plurality of predefined functional states as a function of the alternating electromagnetic field detected.

According to one preferred embodiment, it is provided for that the evaluation unit is further configured to determine an operating state as a functional state of the plasma treatment arrangement as a function of the alternating electromagnetic field detected. In this way, in addition to an error state of the plasma treatment arrangement, the alternating electromagnetic field detected can be used to detect a normal operating state that characterizes error-free operation of the plasma treatment arrangement.

According to one embodiment, it is provided for that the evaluation unit is configured to transform detected measured value progressions, short progressions, of the alternating electromagnetic field detected by means of a Fourier transformation into the frequency space and to determine a functional state of the plasma treatment arrangement in the transformed frequency space.

According to one embodiment, it is intended that the safety device is configured to switch off the high-voltage generator if the evaluation unit has detected an error state as a functional state. To this end, it can be provided for that for safety device has a switch-off device which, when an error state is detected, is separated from the power supply in such a way that the at least one electrode of the electrode arrangement is no longer supplied with an electrical alternating high voltage. In this case, that the electrical connection may be separated, e.g. mechanically, electrically or electronically.

If the safety device detects an error state as a functional state of the plasma treatment arrangement in relation to the electrode arrangement, the high-voltage generator is switched off. In particular, this means that the high-voltage generator is separated from the input voltage. By bringing the plasma treatment arrangement into a safe state, a sustained short circuit with the ground electrode is prevented, especially in the event of a defect in the dielectric.

According to one embodiment, it is intended that the evaluation unit has a machine learning system that contains a learned correlation between measured values of the magnetic field sensors related to the detected alternating electromagnetic field as input data and functional states of the plasma treatment arrangement as output data, wherein the evaluation unit is configured to determine a current functional state of the plasma treatment arrangement as output from the machine learning system as a function of measured values detected by the sensor arrangement as input to the machine learning system.

Such a machine learning system can be, for example, a trained artificial neural network.

The measured values of the magnetic field sensors in relation to the alternating electromagnetic field detected, the former being provided as measured values and originating from the analog values of the magnetic field sensors, act as an input in the machine learning system. The machine learning system has learned a correlation between the measured values and the associated functional state of the plasma treatment arrangement, so that inputting the digital measured values leads to the determination of a corresponding functional state as an output.

To train the machine learning system, especially the artificial neural network, the machine learning system is provided with a range of training data that contains an allocation of measured values or values of the alternating electromagnetic field detected derived from said measured values concerning the respective functional state for which said measured values were detected. For example, a corresponding frequency spectrum of the measured values can be determined for a single measurement series for error-free operation as a functional state, wherein the training data for this measurement sequence then contains the corresponding frequency spectrum with the allocated error-free functional state. Multiple measurement sequences are carried out for each functional state so as to obtain the widest possible spread.

By training the machine learning system with training data provided in this manner, the machine learning system learns a correlation between detected measured values of the alternating electromagnetic field as input data and functional states of the plasma treatment arrangement as output data so that, even in the case of measured values that deviate from the measured values of the training data, a corresponding correct functional state can be determined by the machine learning system.

According to one embodiment, it is provided for that the at least one magnetic field sensor of the sensor arrangement is arranged on the primary or secondary side of the transformer.

According to one embodiment, it is intended that a HALL sensor, an AMR sensor and/or a measuring coil is used as a magnetic field sensor.

Preferably, the plasma treatment arrangement has a control unit which can, but does not have to, contain the safety device. The control unit is preferably designed to apply a pulsed high AC voltage to the electrode arrangement. One pulse duration, i.e. the time in which the pulsed alternating high voltage drops to one twentieth of its maximum value, is preferably at most 1 second, particularly at most 100 ms, preferably at most 10 ms.

The evaluation unit is preferably configured to automatically determine whether a measured value detected by means of the magnetic field sensor, which describes the magnetic field strength of the alternating electromagnetic field, lies within a target interval. If this is not the case, the high-voltage generator is preferably controlled in such a way that it does not apply an alternating high voltage to the electrode arrangement. Alternatively or additionally, a warning is emitted which encodes that there is an error in the electrode arrangement.

The dielectric shields the electrical field acting on the electrode of the electrode arrangement. The thicker the dielectric, the stronger the shielding. The target interval can be open on one side. For example, the target interval can contain a lower limit and be open at the top. If the measured value of the magnetic field strength exceeds the lower limit, there is risk that there is an error in the dielectric.

The evaluation unit preferably comprises a digital memory that contains a target interval for at least one amplitude, especially for at least two amplitudes, especially preferably for multiple amplitudes, of the alternating high voltage. Depending on the amplitude of the alternating high voltage applied to the electrode, the evaluation unit uses the allocated target interval.

The invention is explained in more detail by means of the attached figures. They show

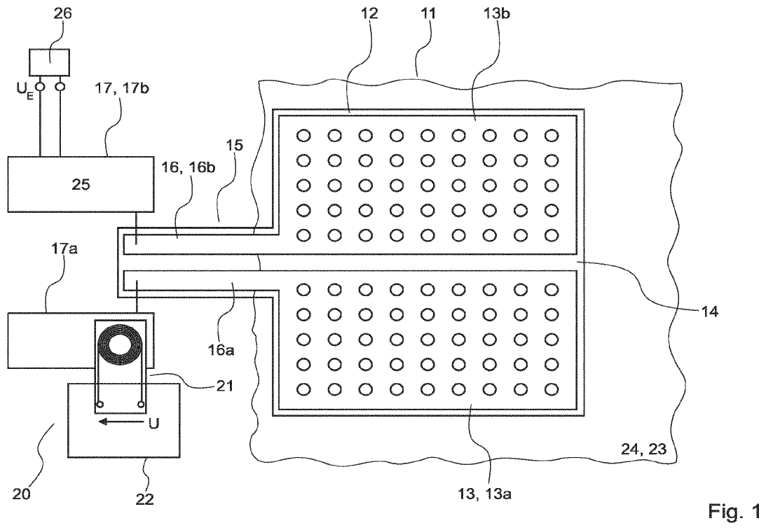

FIG. 1 a schematic representation of an electrode arrangement with two separate partial electrodes;

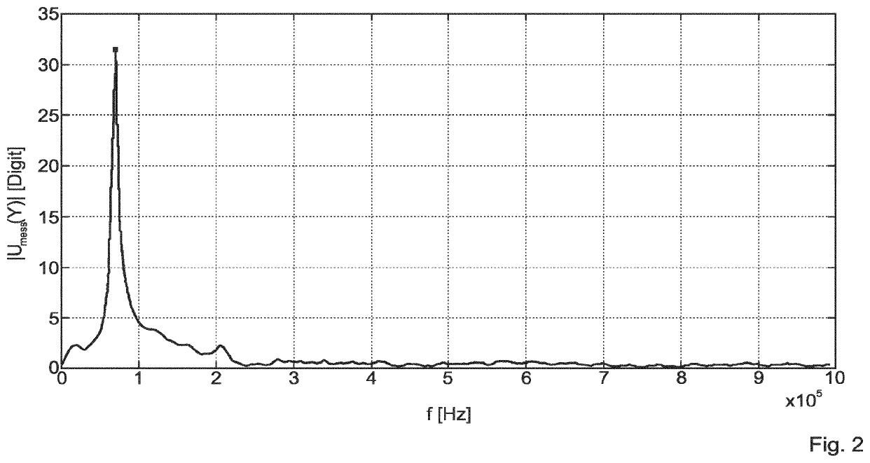

FIG. 2 a frequency spectrum for error-free operation;

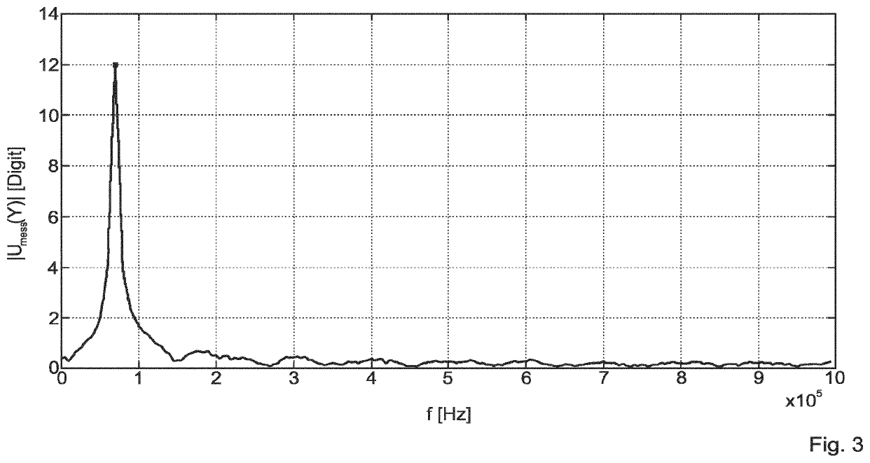

FIG. 3 a frequency spectrum in the event of a one-sided error in the dielectric;

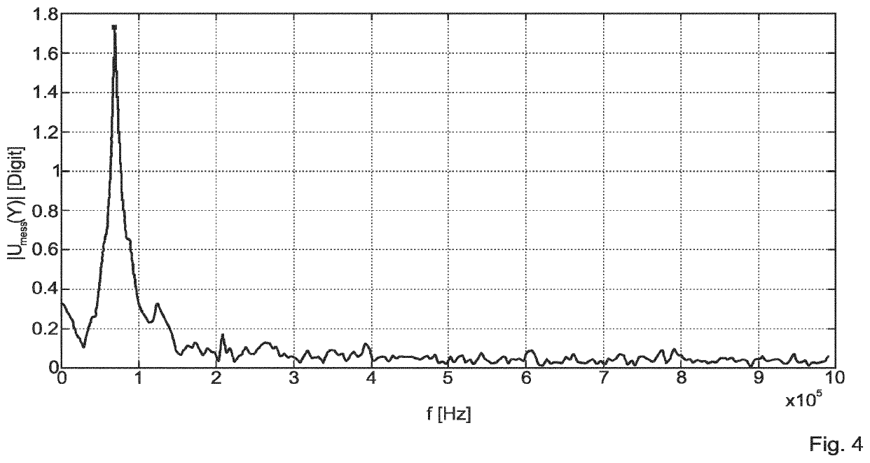

FIG. 4 a frequency spectrum in the event of a two-sided error in the dielectric;

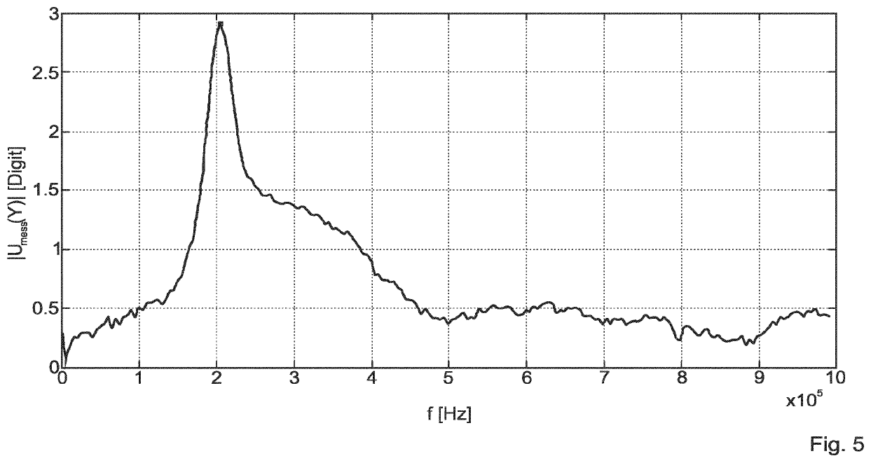

FIG. 5 a frequency spectrum in the event of a short-circuit error;

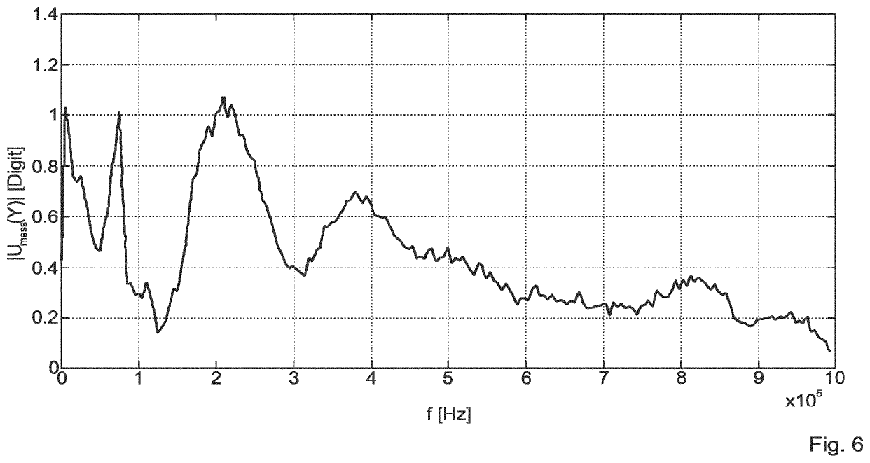

FIG. 6 a frequency spectrum in the event of an idle-state error.

FIG. 1 depicts a plasma treatment arrangement 10 for forming a dielectric barrier plasma discharge. The plasma treatment arrangement 10 has an electrode arrangement 11 with two separate partial electrodes 13 (13a, 13b) embedded in a dielectric 12. The two partial electrodes 13a, 13b are insulated against one another in a center area 14 by the dielectric 12. The two partial electrodes are fed with partial alternating high voltages that compensate each other with regard to the waveform and the voltage level.

In particular, it is provided for that the two partial electrodes do not form a ground electrode (counter electrode) for the respective neighboring partial electrode. Preferably, a surface to be treated 23 forms a ground electrode 24.

The two partial electrodes 13a, 13b can be connected via a connecting piece 15 to an alternating high voltage source in the form of a high-voltage generator 25. The high-voltage generator 25 is supplied with the input voltage U_{E} by a voltage source 26. For each electrode, the connecting piece 15 has an electrically conductive connecting conductor 16, for example a belt-shaped conductor as shown here, with which a high AV voltage U₁₃ₐ, U_{13b} can be applied separately to the respective partial electrode 13a, 13b.

At the end of the connecting piece 15, the connecting conductors 16a for the first partial electrode 13a and 16b for the second partial electrode 13b embedded in the dielectric 12 are contacted by way of a contacting device (not shown), so that the first partial electrode 13a is electrically connected via the connecting conductor 16a of the connecting piece 15 to a first transformer 17 in the form of a trigger transformer 17a, while the second partial electrode 13b is electrically contacted via the connecting conductor 16b of the connecting piece 15 to a second transformer in the form of a trigger transformer 17b. The trigger transformer 17a and the second trigger transformer 17b preferably each form part of the high-voltage generator 25.

With the aid of the trigger transformers 17a, 17b, the input voltage supplied to the alternating high voltage generator U_{E}, which, in particular, can be an input AC voltage, can be transformed into the alternating high voltages U₁₃ₐ, U₁₃ₐ required for the dielectric barrier plasma discharge.

The plasma treatment arrangement 10 also comprises a safety device 20, which has a sensor arrangement 27. The sensor arrangement 27 has a magnetic field sensor 21 as well as an evaluation unit 22.

With the aid of the magnetic field sensor 21, the alternating electromagnetic field generated by the transformer 17 during transformation of the input AC voltage into the desired alternating high voltage for the electrodes is detected. In this way, a measured value progression Uₘ(t) is obtained, for example. The measured value progression is fed to the evaluation unit 22 via a data interface.

The evaluation unit 22 thus obtains a number of measured values Uₘ from the magnetic field sensor 21 that encode, for example, the strength of the magnetic field. The measured values Uₘ can be voltages, but this is not essential. For example, they may also be electric currents. The measured values Uₘ are converted into the frequency spectrum by the evaluation unit 22 by means of an FFT.

On the basis of the frequency spectrum, the evaluation unit 22 can now determine whether a normal, i.e. error-free, operating state or functional state applies or whether there is an error state.

For example, an error state may be a short circuit in the connecting piece 15 between the two connecting conductors 16a, 16b, which differs from the normal operating state by a particular frequency characteristic of the frequency spectrum.

For example, the evaluation unit 22 calculates the square of the deviation of the standardized measured frequency spectrum I(f) from a predetermined target frequency spectrum Iₛₒₗₗ(f). If this squared deviation exceeds a predetermined threshold value, the evaluation unit 22 or a separate control unit operates in such a way that an alternating voltage is no longer applied to the electrode 13.

An error state can also be a so-called idle state in which one or both electrodes on the connecting piece 15 are not properly connected to the respective trigger transformer 17. This often results in a subsequent corona discharge.

An error state can also mean that the dielectric 12 is damaged such that the electrode 12 to be shielded from the dielectric 12 is no longer completely dielectrically shielded by the dielectric 12. If such a defect is not only in one partial electrode 13a, 13b, it often results in a short circuit with the ground electrode 24, which can be determined by the evaluation unit 22 from the frequency spectrum and the specific characteristic for this error state. However, it is also conceivable that the defect of the dielectric 12 is such that both partial electrodes 13a, 13b are affected, resulting in either a short circuit with the ground electrode 24 by way of both partial electrodes 13a, 13b or a short circuit between both partial electrodes 13a, 13b. In this case, too, the corresponding error state can be determined using the specific frequency spectrum of this error.

On the basis of the frequency spectrum of the measured values determined by the magnetic field sensors 21, this frequency spectrum is now allocated a functional state that comes closest to the characteristic of the measured frequency spectrum.

The safety device 20 can be designed in such a way that, in the event of a recognized error state, it cuts the power supply to the electrodes so as to bring the plasma treatment arrangement into a safe state. Particularly when there is a defect in the dielectric, the safety device 20 has to be immediately brought in a safe state in order to further prevent the short circuit with the ground electrode and thus with the surface to be treated under all circumstances.

Here, the evaluation unit 22 may be a data processing device that comprises a measured value interface that allows the analog measured values of the alternating electromagnetic field recorded by the magnetic field sensors 21 to be received. The measured value interface is connected to a measured value amplifier in order to amplify the analog signals in the desired manner. With the aid of a downstream A-D converter, the recorded analog measured values are then converted into digital signals so that the evaluation unit 22 can then evaluate them accordingly.

FIGS. 2 and 6 show a corresponding frequency spectrum for various functional states. Here as in all other cases, one could refer to an amplitude spectrum rather than a frequency spectrum as the amplitude of the corresponding frequency is plotted against the frequency. The data relates to an electrode arrangement that uses conductive silicone as electrode material which is completely embedded in a dielectric.

FIG. 2 depicts the one-sided frequency spectrum calculated by means of FFT in a linear representation of the A-D converted, measured, induced voltage in the measuring coil for normal operation. The target operating state is thus depicted.

The alternating electromagnetic field is detected over time by the magnetic field sensors and converted into an analog electrical measured variable. For example, if the magnetic field sensors are made of an induction coil, an electric voltage can be measured at both ends. Using a suitable electronics assembly (analog-digital conversion), this analog measured variable, such as the detected electric voltage, is converted into a digital signal in which the analog signal is sampled at discrete time intervals. The digital signal, which contains the time characteristic of the analog electrical measured variable (e.g. voltage), is then fed to an FFT evaluation, with which the frequency spectrum shown in FIG. 2 can be calculated as an example.

FIG. 3 depicts the one-sided frequency spectrum calculated by means of FFT in a linear representation of the A-D converted, measured, induced voltage in the measuring coil for the one-sided defect in the dielectric, i.e. the dielectric is defective in the area of just one partial electrode of an electrode arrangement that contains two partial electrodes and thus no longer provides sufficient dielectric shielding.

When directly comparing the frequency spectrum of FIG. 2 in terms of error-free operation with the frequency spectrum of FIG. 3, it is evident that the main frequency is identical, while the difference is in the amplitude whose peak value in FIG. 3 is approximately half the peak value in FIG. 2.

FIG. 4 depicts the one-sided frequency spectrum calculated by means of FFT in a linear representation of the A-D converted, measured, induced voltage in the measuring coil for the two-sided defect in the dielectric, i.e. the dielectric is defective in the area of both partial electrodes of an electrode arrangement that contains two partial electrodes and thus no longer provides sufficient dielectric shielding for both partial electrodes.

Compared to the amplitudes in FIGS. 2 and 3, the main frequency is distinctively narrow-banded and only has an amplitude of fewer than two digits. This means that there is a factor greater than six between the amplitudes in FIGS. 3 and 4. Here, the recognizably more narrow-banded peak of the main frequency can also be evaluated if necessary.

FIG. 5 depicts the one-sided frequency spectrum calculated by means of FFT in a linear representation of the A-D converted, measured, induced voltage in the measuring coil for the short circuit between the partial electrodes of an electrode arrangement comprising two partial electrodes. Compared to the previous amplitude spectra, the main frequency has been displaced and is approximately 200 kHz. This corresponds to a frequency change by a factor of two to three compared with the main frequency during error-free operation.

FIG. 6 depicts the one-sided frequency spectrum calculated by means of FFT in a linear representation of the A-D converted, measured, induced voltage in the measuring coil for idle operation, i.e. no electrode arrangement is electrically contacted. This means that there is a spark or corona discharge, i.e. an electric flashover between the two partial alternating high voltages, in the connection area of the two partial electrodes as the two contacts are open when the electrode arrangement does not cover them and a flashover can occur via the air gap.

A comparison of FIGS. 5 and 6 shows that the main frequency is identical (approximately 200 kHz), but the amplitudes differ by a factor of at least two, almost 3. There are also secondary maxima in the frequency spectrum of FIG. 6. Below 100 kHz, there are frequencies with amplitudes in a similar value range to the amplitude of the main frequency. Information on multiple frequencies with the same intensity can also be included as a differentiation criterion.

Based on the cases described here, at least two parameters are required to differentiate between the cases. This is the amplitude on the one hand and the (main) frequency on the other. The evaluation can, for example, first search for the fundamental frequency and differentiate between them. The information can subsequently used to make a clear determination with respect to the amplitude of the respective status.

Reference list

| | | | |
|---|---|---|---|
| 10 | plasma treatment arrangement | 20 | safety device |
| 11 | electrode arrangement | 21 | magnetic field sensor |
| 12 | dielectric | 22 | evaluation unit |
| 13 | electrode | 23 | surface to be treated |
| 13a | first partial electrode | 24 | ground electrode |
| 13b | second partial electrode | 25 | high-voltage generator |
| 14 | center area | 26 | voltage source |
| 15 | connecting piece | 27 | sensor arrangement |
| 16 | connecting conductor | | |
| 16a | first connecting conductor of the | f | frequency |
| | first partial electrode | i | intensity |
| 16b | second connecting conductor of | I(f) | frequency spectrum |
| | the second partial electrode | t | time |
| 17 | transformer | U₁₃ₐ | high AC voltage |
| 17a | first transformer of the first partial | U_{13b} | high AC voltage |
| | electrode | U_{E} | input voltage |
| 17b | second transformer of the second | Uₘ | measured value |
| | partial electrode | Uₘ(t) | measured value progression |

1. A plasma treatment arrangement for forming a dielectric barrier plasma discharge, comprising:
an electrode arrangement,
comprising at least one electrode and a dielectric embedding the electrode, said dielectric completely covering the electrode towards a surface to be treated:
a high-voltage generator which generates an alternating high voltage by at least one transformer from an input voltage fed to the high-voltage generator, wherein the alternating high voltage is fed to the electrode of the electrode arrangement for forming a dielectric barrier plasma discharge; and
a safety device which comprises
includes a sensor arrangement with at least one magnetic field sensor for detecting an alternating electromagnetic field generated by the at least one transformer, and
an evaluation unit for allocating the alternating electromagnetic field detected by the at least one magnetic field sensor to one of at least two functional states.

2. The plasma treatment arrangement according to claim 1, wherein
(a) the electrode arrangement is designed to be applied to the surface to be treated, and
(b) a functional state is an error state and the evaluation unit is configured to prevent the supply of the alternating high voltage to the electrode arrangement when the error state is detected.

3. The plasma treatment arrangement according to claim 1 wherein the
evaluation unit is configured to detect an error state as a functional state when there is
(a) a short circuit within the electrode of the electrode arrangement, said electrode comprising at least two partial electrodes,
(b) a lack of electrical contacting of the high-voltage generator to the electrode arrangement, and/or
(c) a defect of the dielectric is determined as a function of the alternating electromagnetic field detected.

4. The plasma treatment arrangement according to claim 1 wherein the evaluation unit is configured to determine an error-free operating state as a functional state of the plasma treatment arrangement as a function of the alternating electromagnetic field detected.

5. The plasma treatment arrangement according to claim 1 wherein the evaluation unit is configured to transform detected measured value progressions of the alternating electromagnetic field detected by a Fourier transformation into a frequency spectrum, and to determine a functional state of the plasma treatment arrangement as a function of the transformed frequency spectrum.

6. The plasma treatment arrangement according to claim 1 wherein the evaluation unit is configured to determine a functional state of the plasma treatment arrangement from a plurality of predefined functional states as a function of the alternating electromagnetic field detected.

7. The plasma treatment arrangement according to claim 1 wherein the safety device is configured to switch off the high-voltage generator if the evaluation unit has detected an error state as a functional state.

8. The plasma treatment arrangement according to claim 1 wherein the at least one magnetic field sensor comprises a plurality of magnetic field sensors the evaluation unit comprises a machine learning system that contains a learned correlation between measured values of each of the plurality of magnetic field sensors related to the detected alternating electromagnetic field as input data and functional states of the plasma treatment arrangement as output data, wherein the evaluation unit is configured to determine a current functional state of the plasma treatment arrangement as output from the machine learning system as a function of measured values detected by the sensor arrangement as input to the machine learning system.

9. The plasma treatment arrangement according to claim 8, wherein the machine learning system is a trained artificial neuronal network.

10. The plasma treatment arrangement according to claim 1 wherein the at least one magnetic field sensor of the sensor arrangement is arranged on a primary or a secondary side of the transformer.

11. The plasma treatment arrangement according to one claim 1 wherein the at least one magnetic field sensor is one or more of a HALL sensor, an AMR sensor, and a measuring coil.

12. The plasma treatment arrangement according to claim 1 wherein the plasma treatment arrangement
(a) is designed such that the surface to be treated acts as a ground electrode, and
(b) the safety device is designed such that
a spark-over, and/or
a corona discharge
between the electrode arrangement and the surface to be treated, or between partial electrodes of said electrode is avoided.

13. The plasma treatment arrangement according to claim 1 wherein the plasma treatment arrangement
is designed such that a plasma is generated between the electrode arrangement and the surface to be treated when the electrode arrangement is applied to the surface to be treated and the alternating high voltage is applied to the electrode arrangement.

14. The plasma treatment arrangement according to claim 1 wherein
the evaluation unit is configured to automatically determine whether a measured value detected by the at least one magnetic field sensor, wherein the measured value describes the magnetic field strength of the alternating electromagnetic field, lies within a target interval and, if not, the evaluation unit is configured to control the high-voltage generator such that an alternating high voltage is not applied to the electrode arrangement.
